(19) United States
(12) Patent Application Publication
(10) Pub. No.: US 2025/0251389 A1
(43) Pub. Date: Aug. 7, 2025

(54) Saliva Uric Acid Test Strip Composition and Related Methods to lower elevated levels

(71) Applicant: Shawn J. GREEN, Rockville, MD (US)

(72) Inventor: Shawn J. GREEN, Rockville, MD (US)

(21) Appl. No.: 19/043,985

(22) Filed: Feb. 3, 2025

Related U.S. Application Data
(60) Provisional application No. 63/548,948, filed on Feb. 2, 2024.

(51) Int. Cl. G01N 33/52; A61K 31/353; A61K 31/375; A61K 33/00; A61K 36/736

(52) U.S. Cl. CPC G01N 33/52; A61K 31/353; A61K 31/375; A61K 33/00; A61K 36/736; G01N 2800/04; G01N 2800/32

(57) ABSTRACT

Provided are devices and methods of using such devices for monitoring uric acid levels in an individual comprising the collection of a biological fluid using a test strip and initiating a chemical reaction leading to a color change when the test strip is exposed to the biological fluid containing uric acid. The test strip is an elongated strip with an indent at the mid-point and includes an absorption pad at one end to collect the biological fluid and a test pad at the opposite end embedded with a composition for the chemical reaction. The chemical reaction is a non-enzymatic colorimetric reaction such as a bicinchoninate chelate method.

### CROSS-REFERENCE TO RELATED APPLICATIONS

This application claims priority under 35 U.S.C. § 119 (e) to U.S. Provisional Application No. 63/548,948, filed on Feb. 2, 2024, the entire contents of which are incorporated herein by reference.

### TECHNICAL FIELD

The present disclosure relates to methods and apparatus for monitoring the status of biomarkers. The present disclosure is particularly suited for providing information about an organism's physiological, metabolic, or pathological status. In certain embodiments, the invention herein comprises an user-friendly and convenient apparatus for measuring physiological biomarkers such as metabolites, chemicals, hormones, toxins, enzymes, immunoglobulins, proteins, and nucleic acids, in bodily fluids such as saliva, tears, sweat, urine, and blood. In a particular embodiment, the present invention provides a method, systems and apparatus for monitoring uric acid, and analytes thereof, as well as metabolites in saliva; this information may be utilized as it relates to diets that improve metabolic health, i.e., improving inflammatory conditions and/or kidney disease.

### BACKGROUND OF THE INVENTION

While numerous diagnoses and physical assessments require the use of sophisticated equipment and extensive testing by specialists in clinics, a deeper understanding of biomarkers and their significance provides an opportunity to utilize this information in less obtrusive, less expensive, and less burdensome way. Recent technological advances, along with a better comprehension of metabolic, biochemical and physiological processes have enabled individuals to assume greater responsibility for their own wellness, health and physical fitness. Detection of biomarkers may provide information that can assist individuals in assessing physiological status, and consequently making appropriate adjustments.

Numerous commercial in home medical tests are currently available to the general public, and such tests enable consumers to monitor their health in the privacy of their own homes, without the inconvenience or time-consuming activity of travelling to a healthcare or laboratory facility. Examples of such tests include the pregnancy test (biomarker detected is human chorionic gonadotropin (HCG), test media is urine), blood glucose test (biomarker detected is glucose metabolite, test media is blood), cholesterol test (biomarkers detected include HDL, LDL, and triglycerides, test media is blood), prostate specific antigen (PSA) test (biomarker detected is PSA, test media is blood) as well as COVID-19 test (biomarker detected is SARS-CoV-2 antibody, test media is obtained from nasal or oral specimens). The rising popularity of such tests supports the notion that consumers are becoming increasingly proactive about monitoring various aspects of their health, presumably in an effort to prevent illness and improve quality of life.

Uric acid is a waste product that is produced when the body breaks down purines, which are substances found in certain foods and drinks. High levels of uric acid in the body can lead to several health problems, including gout, kidney stones, and kidney failure. Traditionally, uric acid levels are measured through blood tests, which can be invasive and inconvenient. Furthermore, these tests often require a visit to a healthcare provider and laboratory processing, which can be time-consuming and costly. Therefore, there is a need for a more convenient and less invasive method to monitor uric acid levels. Saliva testing is a non-invasive method that has been used to measure various biomarkers. However, the use of saliva testing for uric acid monitoring is not well established.

It is generally known that a high uric acid level can be the result of the body making too much uric acid, not getting rid of enough of it, or both. Typical causes of a high uric acid level in the blood include: diuretics (water retention relievers), drinking too much alcohol, drinking too much soda or eating too much of foods that contain fructose, genetics, high blood pressure (hypertension), immune-suppressing drugs, kidney problems, leukemia, metabolic syndrome, niacin, also called vitamin B-3, obesity, polycythemia vera, or psoriasis. Additional causes of elevated uric acid include a purine-rich diet, high in foods such as liver, game meat, anchovies and sardines as well as tumor lysis syndrome (a rapid release of cells into the blood caused by certain cancers or by chemotherapy for those cancers).

What is needed are simple, easy-to-use systems and devices that enable users to quickly, and accurately measure biomarkers of interest. In particular, what is needed are tests that can be used to detect biomarkers such as metabolites, chemicals, hormones, toxins, enzymes, immunoglobulins, proteins, and nucleic acids, in bodily fluids such as saliva, tears, sweat, urine, and blood. More particularly, a test that may be used directly in the oral cavity for measuring uric acid status is desired.

### SUMMARY OF THE INVENTION

Provided herein are methods, systems, devices, of uses thereof, for detecting and monitoring physiological uric acid levels and alleviating physiological conditions associated with abnormal levels of uric acid. The devices enable methods for collecting a biological fluid using a test strip, and in cases where the biological fluid contains uric acid, a chemical reaction is initiated leading to a detectable color change. The color change intensifies in proportion to the levels of uric acid being detected.

In accordance with other embodiments, a system is provided for monitoring uric acid. The system comprises a test strip designed to collect a biological fluid and a non-enzymatic colorimetric reaction mechanism embedded in the test strip, which initiates a color change when exposed to the biological fluid containing uric acid.

Through the detection of a saliva analyte, uric acid, an indicator of metabolic disease, the present invention enables individuals to make real-time adjustments to diets and exercise and other lifestyle changes that impact their metabolic health. According to the present invention users are able to rapidly, in a real-time fashion, evaluate and improve their metabolic status by adjusting their intake of foods and beverages that elevate uric acid, for example by reducing alcohol intake, reducing consumption of sodas or other sugary foods, or purine-rich foods. In certain cases, users may opt to seek professional assistance in stabilizing their uric acid levels.

### BRIEF DESCRIPTION OF FIGURES

FIG. 1 provides a flowchart demonstrating operations for monitoring uric acid levels in saliva in accordance with certain embodiments.

FIG. 1A provides a flowchart demonstrating operations for collecting saliva using a specially designed test strip in accordance with certain embodiments.

FIG. 1B provides a flowchart demonstrating operations for initiating a non-enzymatic colorimetric reaction in the test strip when exposed to saliva containing uric acid in accordance with certain embodiments.

FIG. 1C provides a flowchart demonstrating operations for correlating the amount of uric acid in saliva with blood uric acid levels and identifying potential cardiometabolic syndrome in accordance with certain embodiments.

FIG. 2 provides a block diagram illustrating the main components of the Uric Acid Monitoring System.

FIG. 2A provides a block diagram illustrating the ‘Saliva Collection Module’ within the ‘Uric Acid Monitoring System’.

FIG. 2B provides a block diagram demonstrating the ‘Uric Acid Detection Module’ within the ‘Uric Acid Monitoring System’.

FIG. 2C provides a block diagram, the ‘Uric Acid Correlation Module’ within the ‘Uric Acid Monitoring System’.

### DETAILED DESCRIPTION

The present invention may be understood more readily by reference to the following detailed description of the specific embodiments included herein. Reference is made to the accompanying drawings, which form a part hereof, and in which is shown, by way of illustration, various embodiments of the present disclosure. Although the present invention has been described with reference to specific details of certain embodiments thereof, it is not intended that such details should be regarded as limitations upon the scope of the invention.

The entire text of the references mentioned herein are hereby incorporated in their entireties by reference including U.S. Provisional Patent Application Ser. No. 63/548,948 filed on Feb. 2, 2024.

As used herein, the term “subject” should be construed to include subjects, for example medical or surgical subjects, such as humans and other animals requiring therapeutic intervention.

This description of the exemplary embodiments is intended to be read in connection with the accompanying figures, which are to be considered part of the entire written description. In the description, relative terms such as “lower,” “upper,” “horizontal,” “vertical,”, “above,” “below,” “up,” “down,” “top” and “bottom” as well as derivative thereof (e.g., “horizontally,” “downwardly,” “upwardly,” etc.) should be construed to refer to the orientation as then described or as shown in the figure under discussion. These relative terms are for convenience of description and are not considered to be restrictive or limiting.

For purposes of the description hereinafter, it is to be understood that the embodiments described below may assume alternative variations and embodiments. It is also to be understood that the specific articles, compositions, and/or processes described herein are exemplary and should not be considered as limiting.

In the present disclosure the singular forms “a,” “an,” and “the” include the plural reference, and reference to a particular numerical value includes at least that particular value, unless the context clearly indicates otherwise. Thus, for example, a reference to “an antibody” or “an antibody fragment” is a reference to one or more of such structures and equivalents thereof known to those skilled in the art, and so forth. When values are expressed as approximations, by use of the antecedent “about,” it will be understood that the particular value forms another embodiment. As used herein, “about X” (where X is a numerical value) preferably refers to +10% of the recited value, inclusive. For example, the phrase “about 8” preferably refers to a value of 7.2 to 8.8, inclusive; as another example, the phrase “about 8%” preferably (but not always) refers to a value of 7.2% to 8.8%, inclusive. Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, “2-5”, and the like. In addition, when a list of alternatives is positively provided, such listing can be interpreted to mean that any of the alternatives may be excluded, e.g., by a negative limitation in the claims. For example, when a range of “1 to 5” is recited, the recited range may be construed as including situations whereby any of 1, 2, 3, 4, or 5 are negatively excluded; thus, a recitation of “1 to 5” may be construed as “1 and 3-5, but not 2”, or simply “wherein 2 is not included.” It is intended that any component, element, attribute, or step that is positively recited herein may be explicitly excluded in the claims, whether such components, elements, attributes, or steps are listed as alternatives or whether they are recited in isolation.

As used herein, the term “visual intensity” is used in connection with color scales that increase in pigment or hue, for example wherein a scale progresses from a light shade to a dark shade. In certain embodiments, the color scale may increase in hue from whitish pink to deep violet. In certain embodiments, the color scale may increase in hue from whitish yellow to dark brown.

From detecting cancer, to monitoring blood glucose levels and detecting HCG in order to confirm pregnancy, biomarkers have rapidly gained importance as indicators of physiological health. With advances in the identification of specific biomarkers and their role in indicating various physiological or pathological states, there is heightened interest in incorporating the detection of such biomarkers into commercially available, over-the-counter test kits in order to provide consumers with a convenient and cost-effective option for monitoring and maintaining their physical well-being. Such tests have the potential to analyze bodily fluids, including but not limited to, saliva, sputum, tears, sweat, mucus, serum, semen, urine and blood, to detect biomarkers, including but not limited to, analytes, metabolites, chemicals, hormones, toxins, enzymes, immunoglobulins, proteins, and nucleic acids. Ideally such tests are disposable and/or biodegradable.

The confluence of increasing health care costs together with increasing knowledge concerning the causality of many health conditions, mandates a prudent approach to monitoring factors that cause illness or lead to poor physiological status. The use of information concerning biomarkers may be incorporated into tests that allow individuals to follow their health and well-being and to make adjustments to their lifestyle (i.e. diet and exercise) as necessary. Just as glucose monitors have been instrumental in enabling diabetic patients to monitor blood sugar levels and thereby manage their healthcare, there exists tremendous potential and need for tests that utilize other biomarkers to better maintain health. Biomarkers can be useful in predicting risk, screening, diagnosis, scaling severity, monitoring progress, predicting response to therapy, determining prognosis and understanding disease mechanism.

The present disclosure comprises the use of biomarker information in easy-to-use tests in order to provide individuals with useful information about their health and to enable individuals to take appropriate action to alleviate problematic levels of metabolites and the like. The tests included herein involve the analysis of bodily fluids to detect a variety of biomarkers. Body fluids that may be analyzed herein, include but are not limited to, saliva, sputum, tears, sweat, mucus, serum, semen, urine and blood.

Recent studies have identified uric acid as an emerging biomarker for the onset of unhealthy dietary lifestyle: persistent and elevated levels of uric acid may be an early sign for metabolic problems. Proper hydration and replacing sugar-laden and purine-rich foods with plant-based diets may be an effective way to combat uric acid. As described herein, the unique salivary test trips of the invention enable users to easily monitor uric acid levels and subsequently modify their diet so as to reduce the harmful effects of elevated uric acid. Dietary modifications may comprise increasing the intake of plant-based and alkalinizing foods, and the reduction of purine-rich foods (such as red meat and certain seafood), alcohol, and excessive consumption of High Fructose Corn Syrup (HFCS)-loaded sodas and snacks, which can contribute to weight gain and long-term cardiovascular complications. Such complications are clustered under cardio-metabolic syndrome, a combination of risk factors including abdominal obesity, hypertension, impaired fasting blood glucose, high levels of triglycerides, and low levels of HDL that, collectively, putting individuals at risk for heart disease and diabetes. Replacing purine-rich foods and alcohol consumption with plant-based diets, such as the Mediterranean, MIND, and/or DASH Diet and rehydrating with alkalinizing foods, including lemon juice and apple cider vinegar, are believed to constitute effective intervention for addressing elevated uric acid levels. Certain vegetables and fruits, such as, tart cherry and celery seed are also reportedly effective at reducing uric acid levels.

A significant positive correlation between the concentrations of uric acid in saliva and blood has been established by multiple, independent labs. In a report on 78 volunteers between the ages of 18-65, Soukup et al (2012) observed a linear relationship between salivary and serum uric acid concentrations and a significant elevation in salivary uric acid concentration, independent of salivary flow rate, in patients with metabolic syndrome. Of particular significance was the correlation seen between salivary uric acid and a number of markers reflective of poor dietary lifestyle. Total body uric acid is a balance between production and elimination. Approximately ⅔ of uric acid produced each day is excreted in urine and ⅓ eliminated directly in saliva and intestinal secretion. Owen-Smith et al (1998) first suggested salivary uric acid sampling as a non-invasive indicator for monitoring purine metabolism. A linear relationship was observed between serum and saliva uric acid. (Owen-Smith et al. 1998). Similarly, Schermann et al. (1977) demonstrated that salivary secretion is predictive of serum concentrations in both normal and hyperuricemic subjects. Ping et al. (2013) reported a correlation coefficient between serum and saliva of 0.948 (p<0.05) further reinforcing similar observations of Goll and Mookerjee (1978), Blicharz et al. (2008), Passos et al. (2012). Based on 191 participants, Shibasaki et al. (2012) independently confirmed a positive correlation between the uric acid concentrations in serum and saliva (r=0.503, P<0.01) with serum levels of 6.31±0.24 mg/dl (375±14.28 uM) proportionally higher by a factor of 1.8 to saliva levels of 3.38±0.21 mg/dl (201±12.49 uM). When subjects were divided into two groups based on serum levels less (Normal) than and greater (Elevated) than 7 mg/dl (416 uM), a similar pattern emerged as to the ratio of serum-to-saliva concentration in Normal (1.79) and Elevated (1.94) within each cohort. As described herein, using a non-invasive, self-check saliva-based testing approach enables the overcoming of barriers to regular screening and improves adherence to dietary and behavioral treatment programs.

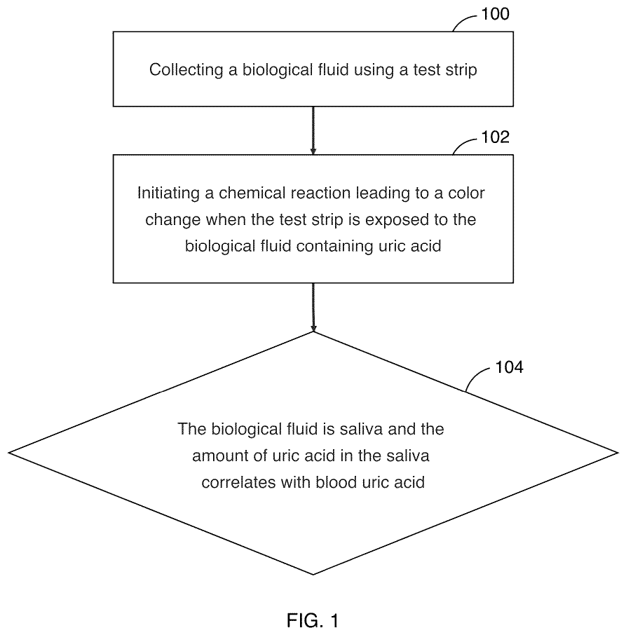

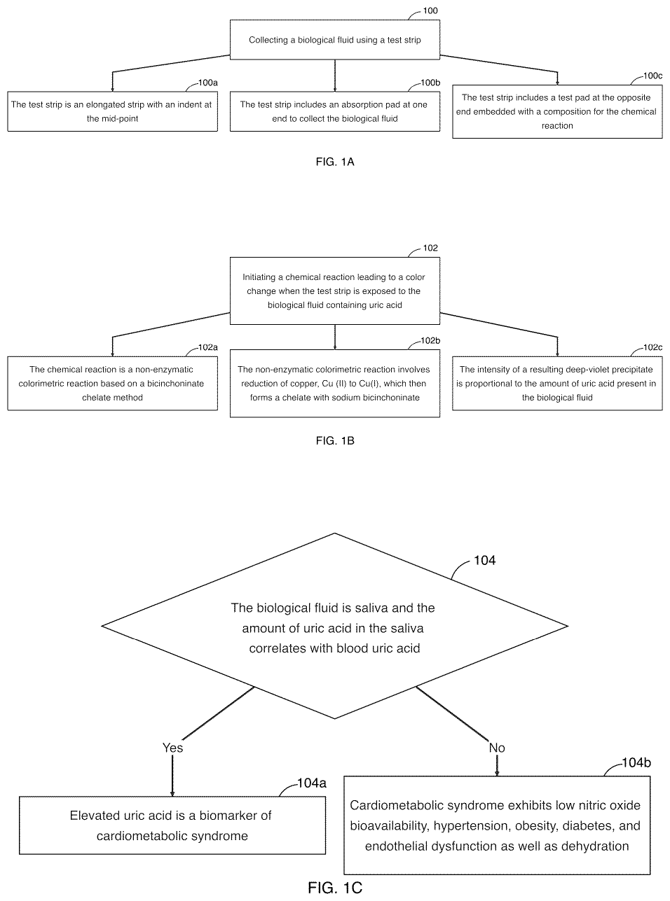

Provided herein are devices and methods of use thereof for monitoring and modifying uric acid: in an embodiment, as summarized in FIG. 1, the device comprises a test strip designed to collect a biological fluid. The biological fluid, initiates a chemical reaction on the test strip leading to a color change on the test strip if uric acid is detected in biological fluid. The intensity of the color change increases in proportion the levels of uric acid in the biological fluid. In an embodiment, the biological fluid comprises saliva. As described above, the levels of uric acid in the saliva correlates with the levels of uric acid in blood.

As shown in FIG. 1A the test strip, further detailed in sub-steps 100-a, 100-b, and 100-c, is an elongated strip with an indent at the mid-point. This design allows for the folding of the strip, enabling the opposite ends of the strip to make contact when pressed together. One end of the strip has an absorption pad, used to collect the saliva directly from the mouth. This pad absorbs the saliva, ensuring a sufficient sample size for the subsequent testing process. The opposite end of the strip includes a test pad, embedded with a composition for initiating a chemical reaction. This composition reacts with the uric acid present in the saliva sample, leading to a color change that indicates the uric acid level. The saliva, the test strip, the absorption pad, and the test pad are all involved in this step. Their specific design and function contribute to the accuracy and reliability of the uric acid monitoring method.

Step 102 (FIG. 1B) and its sub-steps describe the initiation of a chemical reaction that results in a color change when the test strip comes into contact with saliva containing uric acid. The chemical reaction, detailed in sub-step 102-a, is a non-enzymatic colorimetric reaction based on a bicinchoninate chelate method. This reaction does not require an enzyme, which simplifies the testing process. In sub-step 102-b, the non-enzymatic colorimetric reaction involves the reduction of copper, Cu (II) to Cu(I), which then forms a chelate with sodium bicinchoninate. This reaction is specific to the presence of uric acid in the saliva sample. In sub-step 102-c, the intensity of the resulting deep-violet precipitate is proportional to the amount of uric acid present in the saliva. This provides a measurable way to determine uric acid levels. The test strip, the chemical reaction, and the resulting precipitate are all involved in this step. Their functions and interactions contribute to the testing process.

Step 104 (FIG. 1C) and its sub-steps describe the identification of the biological fluid as saliva and the correlation of the amount of uric acid in the saliva with blood uric acid levels. The biological fluid is saliva, as detailed in Step 104. Saliva is a readily available and easily collected sample, making it a practical choice for a test that may be used frequently or by individuals without medical training. The amount of uric acid in the saliva correlates with blood uric acid levels, as detailed in Step 104. By measuring the uric acid levels in saliva, it is possible to get an indication of the uric acid levels in the blood. Sub-step 104-a identifies elevated uric acid as a biomarker of cardiometabolic syndrome. High levels of uric acid in the saliva, and by extension in the blood, can be an indicator of this condition. Sub-step 104-b elaborates on the characteristics of cardiometabolic syndrome, which include low nitric oxide bioavailability, hypertension, obesity, diabetes, and endothelial dysfunction as well as dehydration. The saliva, the uric acid, and the cardiometabolic syndrome are all involved in this step. Their functions and interactions contribute to the testing process.

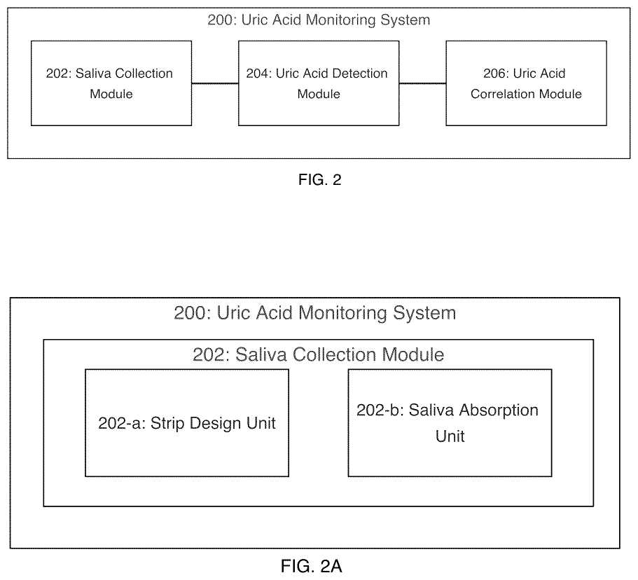

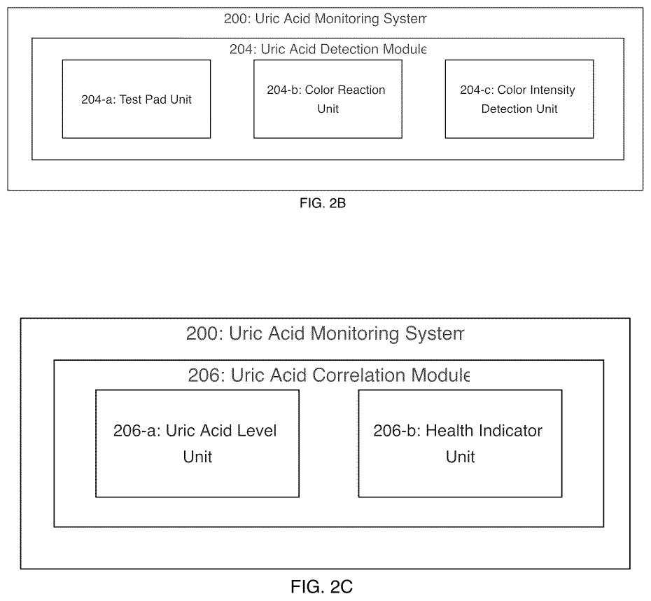

In an embodiment as shown in FIG. 2, the Uric Acid Monitoring System, numbered as 200, is designed to monitor uric acid levels in the body. This system aids in the early detection and management of conditions such as gout and kidney stones, which are often associated with elevated uric acid levels. The system is composed of several key components (FIG. 2A) The Saliva Collection Module, numbered as 202, includes the Strip Design Unit (202-a) and the Saliva Absorption Unit (202-b). The Strip Design Unit refers to the test strip's design, which is elongated with an indent at the mid-point. This design facilitates the collection of saliva, a biological fluid, directly from the mouth. The Saliva Absorption Unit refers to the absorption pad located at one end of the test strip. This pad is designed to collect and hold the saliva sample for testing.

The Uric Acid Monitoring System operates through a series of steps. The process begins with the Saliva Collection Module. The unique design of the test strip, part of the Strip Design Unit, allows it to be easily folded and placed in the mouth. Once the strip is in place, the Saliva Absorption Unit comes into play. The pad is designed to efficiently collect and hold the saliva sample for testing. The saliva is collected directly from the mouth, providing a non-invasive method of sample collection. The collected saliva sample is expected to contain uric acid, which is the primary substance of interest in this system. The level of uric acid in the saliva is indicative of the uric acid level in the body, making saliva an effective biological fluid for this test. The design and functionality of these components ensure that the saliva sample is collected effectively and efficiently, setting the stage for the subsequent steps of uric acid detection and correlation. The process is designed to be user-friendly and non-invasive, making it suitable for regular monitoring of uric acid levels. The accuracy and reliability of the results are ensured by the careful design and coordination of the various components and their associated actions.

The Saliva Collection Module, denoted as 202, is part of the Uric Acid Monitoring System. It plays a role in the initial stage of the monitoring process, which involves the collection of saliva directly from the mouth.

This module is composed of two units. The first is the Strip Design Unit, numbered as 202-a. This unit refers to the design of the test strip, which is elongated and features an indent at the mid-point. This design allows the strip to be folded and placed in the mouth, facilitating the collection of saliva.

The second unit is the Saliva Absorption Unit, numbered as 202-b. This unit refers to the absorption pad located at one end of the test strip. The pad is designed to collect and hold the saliva sample for testing.

The Saliva Collection Module operates through a series of steps. The process begins with the Strip Design Unit. The design of the test strip allows it to be folded and placed in the mouth. Once the strip is in place, the Saliva Absorption Unit comes into play. The pad collects and holds the saliva sample for testing. The saliva is collected directly from the mouth, providing a non-invasive method of sample collection. The collected saliva sample is expected to contain uric acid. The level of uric acid in the saliva is indicative of the uric acid level in the body, making saliva an effective biological fluid for this test. The design and functionality of these units ensure that the saliva sample is collected effectively and efficiently, setting the stage for the subsequent steps of uric acid detection and correlation. The process is designed to be user-friendly and non-invasive, making it suitable for regular monitoring of uric acid levels. The results are ensured by the design and coordination of the various units and their actions.

The Uric Acid Detection Module (FIG. 2B), denoted as 204, is part of the Uric Acid Monitoring System. It plays a role in the detection of uric acid levels in the saliva sample collected by the Saliva Collection Module. This module is composed of three units. The first is the Test Pad Unit, numbered as 204-a. This unit refers to the test pad located at the opposite end of the test strip. The pad is embedded with a composition that reacts chemically when exposed to saliva containing uric acid.

The second unit is the Color Reaction Unit, numbered as 204-b. This unit refers to the non-enzymatic colorimetric reaction that takes place when the test pad is exposed to saliva containing uric acid. This reaction involves the reduction of copper, Cu (II) to Cu(I), which then forms a chelate with sodium bicinchoninate. The third unit is the Color Intensity Detection Unit, numbered as 204-c. This unit refers to the resulting deep-violet precipitate that is formed as a result of the colorimetric reaction. The intensity of this precipitate is proportional to the amount of uric acid present in the saliva, providing a visual indication of uric acid levels.

The Uric Acid Detection Module operates through a series of steps. The process begins with the Test Pad Unit. The pad is embedded with a composition that reacts when exposed to saliva containing uric acid. Once the saliva sample is applied to the test pad, the Color Reaction Unit comes into play. This reaction involves the reduction of copper, Cu (II) to Cu(I), which then forms a chelate with sodium bicinchoninate. The resulting deep-violet precipitate is then analyzed by the Color Intensity Detection Unit. The intensity of this precipitate is proportional to the amount of uric acid present in the saliva, providing a visual indication of uric acid levels. The design and functionality of these units ensure that uric acid levels in the saliva are accurately detected, setting the stage for the subsequent steps of uric acid correlation. The process is designed to be user-friendly and non-invasive, making it suitable for regular monitoring of uric acid levels. The results are ensured by the design and coordination of the various units and their actions.

The Uric Acid Correlation Module (FIG. 2C), denoted as 206, is part of the Uric Acid Monitoring System. It plays a role in correlating the detected uric acid levels with various health indicators. This module is composed of two units. The first is the Uric Acid Level Unit, numbered as 206-a. This unit refers to the uric acid levels detected in the saliva sample. The level of uric acid in the saliva is indicative of the uric acid level in the body, making saliva an effective biological fluid for this test. The second unit is the Health Indicator Unit, numbered as 206-b. This unit refers to the various health indicators that are associated with uric acid levels. These include conditions such as cardiometabolic syndrome, which exhibits low nitric oxide bioavailability, hypertension, obesity, diabetes, and endothelial dysfunction as well as dehydration.

The Uric Acid Correlation Module operates through a series of steps. The process begins with the Uric Acid Level Unit. The level of uric acid in the saliva is indicative of the uric acid level in the body. Once the uric acid levels are detected, the Health Indicator Unit comes into play. This unit refers to the various health indicators that are associated with uric acid levels. The detected uric acid levels are then correlated with these health indicators to provide a comprehensive understanding of the individual's health status. The process is designed to be user-friendly and non-invasive, making it suitable for regular monitoring of uric acid levels. The results are ensured by the design and coordination of the various units and their actions. The design and functionality of these units ensure that the correlation of uric acid levels with health indicators is accurately determined, setting the stage for the subsequent steps of health management and intervention.

Disclosed herein are methods for decreasing physiological uric acid levels comprising: a first step of monitoring uric acid, comprising; collecting a biological fluid using a test strip; initiating a chemical reaction leading to a color change when the test strip is exposed to the biological fluid containing uric acid; wherein the test strip is an elongated strip with an indent at the mid-point, including an absorption pad at one end to collect the biological fluid and a test pad at the opposite end embedded with a composition for the chemical reaction; and, a second step comprising implementing dietary modifications. In an embodiment, the chemical reaction is a non-enzymatic colorimetric reaction and in certain embodiments, the non-enzymatic colorimetric reaction is based on a bicinchoninate chelate method. The non-enzymatic colorimetric reaction may involve reduction of copper, Cu (II) to Cu(I), which then forms a chelate with sodium bicinchoninate. Furthermore, the intensity of the non-enzymatic colorimetric reaction may be proportional to the amount of uric acid present in the biological fluid, with a resolution and range of the following values in mg/dL: 1.68, 2.56, 3.36, 4.20, 5.04, 5.88, 46.72, 7.57. As contemplated herein, the methods of the invention comprise methods wherein the biological fluid is saliva and the amount of uric acid in the saliva correlates with blood uric acid. Elevated uric acid, is considered to be approximately greater that 4 mg/dL. In an embodiment, the chemical reaction is a non-enzymatic colorimetric reaction that increases in visual intensity and corresponds to increasing levels of uric acid levels such as 150 mM, 200 μM, 250 μM, 300 μM, 400 μM, 450 μM and 750 μM per sample. As contemplated herein, the methods of the invention are useful for identifying metabolic and cardiometabolic syndromes such as those including, but not limited to, gout, kidney disease, kidney stones, inflammation, low nitric oxide bioavailability, hypertension, arthritis obesity, diabetes, endothelial dysfunction, heart disease, fatty liver, dehydration and/or musculoskeletal problems.

In an embodiment, upon detection of elevated levels of uric acid, a user may respond by implementing dietary modifications, by increasing the consumption of Vitamin C, potassium nitrate, tart cherry extract, quercetin, leafy greens, and/or decreasing the consumption of purine-rich foods and/or beverages, red meat, organ meats, seafood, alcohol and/or high sucrose or fructose containing food and beverages. In an embodiment, dietary modification may comprise ingestion of a composition comprising a blend of 20-1,000 mg Vitamin C, 25-300 mg potassium nitrate, 100-500 mg tart cherry extract (10:1) and 200-1,000 mg quercetin.

In an embodiment, provided herein are systems and devices for monitoring uric acid, comprising: a test strip designed to collect a biological fluid; a non-enzymatic colorimetric reaction mechanism embedded in the test strip, which initiates a color change when exposed to the biological fluid containing uric acid, wherein the test strip is an elongated strip with an indent at the mid-point, wherein the test strip includes an absorption pad at one end to collect the biological fluid, wherein the test strip includes a test pad at the opposite end embedded with a composition for the chemical reaction; and wherein the chemical reaction is a non-enzymatic colorimetric reaction. The test-strips contemplated herein may be disposable, and/or biodegradable.

### REFERENCES

M. Soukup, I. Biesiada, A. Henderson, B. Idowu, D. Rodeback, L. Ridpath, et al., Salivary uric acid as a noninvasive biomarker of metabolic syndrome. Diabetol. Metab. Syndr. 4 (2012) 14.
Owen-Smith, Brian, Jeremy Quiney, and James Read. “Salivary urate in gout, exercise, and diurnal variation.” The Lancet 351.9120 (1998): 1932.
Schermann, J. M., et al. “Comparative study of uric acid concentration in serum and saliva of healthy or hyperuricemic subjects.” Annales de Biologie Clinique. Vol. 35. No. 6. 1977.
Ping, C et al. 2013. J Cent South Univ (Med Sci) 38:1260
Dai X, Yuan J, Yao P, Yang B, Gui L, Zhang X, Guo H, Wang Y, Chen W, Wei S, Miao X, Li X, Min X, Yang H, Fang W, Liang Y, Hu F B, Wu T, He M. Association between serum uric acid and the metabolic syndrome among a middle- and old-age Chinese population. Eur J Epidemiol. 2013 August; 28 (8): 669-76. doi: 10.1007/s10654-013-9829-4. Epub 2013 Jul. 18. PMID: 23864233.
Goll R D, Mookerjee B K. Correlation of biochemical parameters in serum and saliva in chronic azotemic patients and patients on chronic hemodialysis. J Dial. 1978; 2 (4): 399-44. doi: 10.3109/08860227809079326. PMID: 721995.
Timothy M Blicharz, David M Rissin, Michaela Bowden, Ryan B Hayman, Christopher DiCesare, Jasvinder S Bhatia, Nerline Grand-Pierre, Walter L Siqueira, Eva J Helmerhorst, Joseph Loscalzo, Frank G Oppenheim, David R Walt, Use of Colorimetric Test Strips for Monitoring the Effect of Hemodialysis on Salivary Nitrite and Uric Acid in Patients with End-Stage Renal Disease: A Proof of Principle, Clinical Chemistry, Volume 54, Issue 9, 1 Sep. 2008, Pages 1473-1480.
Passos et al., 2012. J. Biosensor & Bioelectronics 3:5

1. A method for decreasing uric acid comprising:
a. a first step of monitoring uric acid, comprising;
collecting a biological fluid using a test strip;
initiating a chemical reaction leading to a color change when the test strip is exposed to the biological fluid containing uric acid;
wherein the test strip is an elongated strip with an indent at the mid-point, including an absorption pad at one end to collect the biological fluid and a test pad at the opposite end embedded with a composition for the chemical reaction; and,
b. a second step comprising implementing dietary modifications.

2. The method of claim 1, wherein the chemical reaction is a non-enzymatic colorimetric reaction based on a bicinchoninate chelate method.

3. The method of claim 2, wherein the non-enzymatic colorimetric reaction involves reduction of copper, Cu (II) to Cu(I), which then forms a chelate with sodium bicinchoninate.

4. The method of claim 3, wherein the intensity of the non-enzymatic colorimetric reaction is proportional to the amount of uric acid present in the biological fluid, with a resolution and range of the following values in mg/dL: 1.68, 2.56, 3.36, 4.20, 5.04, 5.88, 46.72, 7.57.

5. The method of claim 4, wherein the biological fluid is saliva and the amount of uric acid in the saliva correlates with blood uric acid.

6. The system of claim 5, wherein elevated uric acid, as defined by >4 mg/dL, is a biomarker of metabolic and cardiometabolic syndromes.

7. The method of claim 1, wherein the chemical reaction is a non-enzymatic colorimetric reaction that increases in visual intensity and corresponds to increasing levels of uric acid levels such as 150 μM, 200 μM, 250 μM, 300 μM, 400 μM, 450 μM and 750 μM per sample.

8. The method of claim 6, wherein metabolic and cardiometabolic syndromes comprise gout, kidney disease, kidney stones, inflammation, low nitric oxide bioavailability, hypertension, arthritis obesity, diabetes, endothelial dysfunction, heart disease, fatty liver, dehydration and/or musculoskeletal problems.

9. The method of claim 1, wherein the dietary modifications comprise:
increasing the consumption of Vitamin C, potassium nitrate, tart cherry extract, quercetin, leafy greens,
and/or decreasing the consumption of purine-rich foods and/or beverages, red meat, organ meats, seafood, alcohol and/or high sucrose or fructose containing food and beverages.

10. The method of claim 9, wherein the dietary modification comprises ingestion of a composition comprising a blend of 20-1,000 mg Vitamin C, 25-300 mg potassium nitrate, 100-500 mg tart cherry extract (10:1) and 200-1,000 mg quercetin.

11. A system for monitoring uric acid, comprising:
a. a test strip designed to collect a biological fluid; a non-enzymatic colorimetric reaction mechanism embedded in the test strip, which initiates a color change when exposed to the biological fluid containing uric acid, wherein the test strip is an elongated strip with an indent at the mid-point, wherein the test strip includes an absorption pad at one end to collect the biological fluid, wherein the test strip includes a test pad at the opposite end embedded with a composition for the chemical reaction; and
b. wherein the chemical reaction is a non-enzymatic colorimetric reaction.

12. The system of claim 11, wherein the non-enzymatic colorimetric reaction is based on a bicinchoninate chelate method and involves reduction of copper, Cu (II) to Cu(I), which then forms a chelate with sodium bicinchoninate.

13. The system of claim 12, wherein the intensity of the non-enzymatic colorimetric reaction is proportional to the amount of uric acid present in the biological fluid, with a resolution and range of the following values in mg/dL: 1.68, 2.56, 3.36, 4.20, 5.04, 5.88, 46.72, 7.57.

14. The system of claim 13, wherein the biological fluid is saliva and the amount of uric acid in the saliva correlates with blood uric acid.

15. The system of claim 14, wherein elevated uric acid, as defined by >4 mg/dL, is a biomarker of metabolic or cardiometabolic syndrome.

16. The system of claim 15, wherein metabolic and cardiometabolic syndromes comprise gout, kidney disease, kidney stones, inflammation, low nitric oxide bioavailability, hypertension, arthritis obesity, diabetes, endothelial dysfunction, heart disease, fatty liver, dehydration and/or musculoskeletal problems.

17. A system of claim 11, further comprising a step of dietary modification upon detection of elevated uric acid.

18. The system of claim 17, wherein the dietary modifications comprise:
increasing the consumption of Vitamin C, potassium nitrate, tart cherry extract, quercetin, leafy greens,
and/or decreasing the consumption of purine-rich foods and/or beverages, red meat, organ meats, seafood, alcohol and/or high sucrose or fructose containing food and beverages.

19. The system of claim 18, wherein the dietary modification comprises ingestion of a composition comprising a blend of 20-1,000 mg Vitamin C, 25-300 mg potassium nitrate, 100-500 mg tart cherry extract (10:1) and 200-1,000 mg quercetin.

20. The system of claim 11, wherein the non-enzymatic colorimetric reaction increases in visual intensity and corresponds to increasing levels of uric acid levels such as 150 μM, 200 μM, 250 μM, 300 μM, 400 μM, 450 μM and 750 μM per sample.
